(19) United States
(12) Patent Application Publication
(10) Pub. No.: US 2010/0056483 A1
(43) Pub. Date: Mar. 4, 2010

(54) METHODS AND COMPOSITIONS FOR THE TREATMENT AND PREVENTION OF BONE LOSS

(75) Inventors: Yang V. Li, Athens, OH (US); Caleb O. Molokwu, Dayton, OH (US)

Correspondence Address: CALFEE HALTER & GRISWOLD, LLP, 800 SUPERIOR AVENUE, SUITE 1400, CLEVELAND, OH, 44114, US

(73) Assignee: OHIO UNIVERSITY, Athens, OH (US)

(21) Appl. No.: 12/429,570

(22) Filed: Apr. 24, 2009

Related U.S. Application Data
(60) Provisional application No. 61/093,702, filed on Sep. 2, 2008.

(51) Int. Cl. A61K 31/663; A61K 31/565; A61K 31/566; A61K 31/65; A61K 31/47; A61K 31/44; A61P 19/10; A61P 19/08

(52) U.S. Cl. 514/108; 514/171; 514/154; 514/311; 514/357

(57) ABSTRACT

Disclosed herein are methods and compositions for the treatment and prevention of bone loss. The methods comprise providing a therapeutically effective amount of at least one chelating agent to a subject. The methods further comprise providing a therapeutically effective amount of estrogen or at least one estrogen analogue to a subject. Compositions disclosed herein for the treatment and prevention of bone loss comprise a chelator and estrogen or at least one estrogen analogue. The compositions further comprise at least one of a bisphosphonate, a selective estrogen receptor modulator, or a hormone.

### CROSS-REFERENCE TO RELATED APPLICATION

This application claims priority to and any other benefit of U.S. Provisional Application Ser. No. 61/093,702, filed on Sep. 2, 2008, the entire content of which is incorporated by reference herein.

### BACKGROUND

Zinc, as a dietary component or supplement, is important to bone health throughout life. While many studies have demonstrated the signaling role of zinc in biological system, some of zinc's regulatory action may not be in the interest of osteoporosis or hormone therapy. Studies have shown that zinc inhibits the aromatase enzyme that converts testosterone into excess estrogen. Zinc can negatively regulate the availability of parathyroid hormone. Both hormones play a major role in bone remodeling. They can help prevent and treat osteoporosis. Studies have also shown zinc's ability to compete with the estrogen transport via sex hormone-binding globulin (SHBG), a protein that transports estrogen in the blood. SHBG has been shown to have specific binding sites for zinc. Zinc binding to a site at the entrance of the steroid-binding pocket in human SHBG has been shown to reduce the affinity of SHBG for estrogen.

### SUMMARY

In one embodiment, the invention is a method for preventing bone loss by providing an individual in need with a therapeutically significant dose of a zinc chelator. In one such embodiment, the bone loss to be prevented is osteoporosis.

In another embodiment, the invention is a method for treating bone loss by providing an individual in need with a therapeutically significant dose of a zinc chelator and estrogen, or an estrogen analogue. In one such embodiment, the bone loss to be prevented is osteoporosis.

In another embodiment, the invention is a method for treating bone loss by providing an individual in need with a therapeutically significant dose of a chelating agent and estrogen, or an estrogen analogue. In one such embodiment, the bone loss to be prevented is osteoporosis.

Additional features and advantages will be set forth in part in the description that follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF FIGURES

The accompanying drawings, which are incorporated in and constitute a part of this specification, illustrate some embodiments of the invention, and together with the description, serve to explain principles of the invention.

FIG. 1: Diagram showing zinc inhibits the conversion of testosterone to estrogen.

FIG. 2: Model diagram showing zinc competing with estrogen on the sex hormone binding globulin receptor.

FIG. 3: Schematic representation of the effect of zinc on ligand-receptor systems in osteoclast differentiation and the interaction between osteoblast and osteoclast, showing the effect of zinc coupled with estrogen on osteoclast and osteoblast signaling. The RANKL/RANK interaction is responsible for development and differentiation of osteoclast cells. The osteoblast cell in the presence of zinc and estrogen stimulates production of TGF-β, which favors increased production of OPG, a protein that can be secreted outside the cell and then bind RANKL and prevent it from interacting with RANK, thus blocking the formation and activation of osteoclasts. The balance between RANKL and OPG determines how fast bone breaks down. (Molokwu & Li. 2006 Ohio Research and Clinical Review 15:7-15)

FIG. 4: An Ohio University MicroCT suitable for small animals (in vivo or in vitro), fossils, and other objects smaller than about 10 cm diameter to be non-destructively imaged and digitized in 3D at resolutions of 92, 45, and 27 microns. Microcomputer tomography (microCT) is a miniaturized version of computerized axial tomography commonly used by radiologists but the systems have a resolution on the order of a few micrometers. The technique is now favored in the study of trabecular bone loss in osteoporotic patients or in animal models of osteoporosis (Ryan T M & Ketcham R A, Journal of Human Evolution (2002) 43,1-26).

FIG. 5 (A and B): Diagram showing a representation of control and experimental rats from left to right after 10 weeks of treatment: Group A (Non-OVX) Control, Group B (OVX), Group C (OVX+E2), Group D (OVX+CaEDTA), and Group E (OVX+E2+CaEDTA).

FIG. 6 (A and B): Differences in weight between the control group and the treatment groups.

FIG. 7: Scanned Femur bone showing differences in trabeculation of the metaphysis of the control and treatment groups. A, Group A sham control. B, Group B OVX control. C, Group C OVX+estradiol. D, Group D OVX+CaEDTA. E, Group E OVX+estradiol+CaEDTA.

FIG. 8: BMD measurement of (A) Entire femur, (B) Clip of Range of interest of midshaft, (C) Proximal femur, (D) Midshaft of the femur, (E) Distal femur and (F) OVX rat with porous femur bone, using the Microview software.

FIG. 9: Scanned Femur bone showing (i) Caudal, (ii) Cranial, (iii) Lateral, (iv) Medial, (v) Coronal section, (vi) Sagitttal section, and (vii) Transverse section.

FIG. 10: Graphical analysis of the femur bone showing difference in bone mineral density of five different rat groups, after completed 8 and 12 weeks of treatments. Values are expressed as the mean±SEM. *, P<0.05 vs. OVX controls. A, Group A sham control. B, Group B OVX control. C, Group C OVX+estradiol. D, Group D OVX+CaEDTA. E, Group E OVX+estradiol+CaEDTA.

FIG. 11 (A and B): A. TBMD of each group is compared to Group B (reference group). Values are the accumulated percentage increases measured at 8 and 12 weeks. B. The TBMD increases of group C, D and E are expressed as the proportional increases of sum TBMD increases (100%) from three groups. The percentage increase in Group E (estradiol+CaEDTA) are larger (>60%) than the sum combined increases in Group C and D.

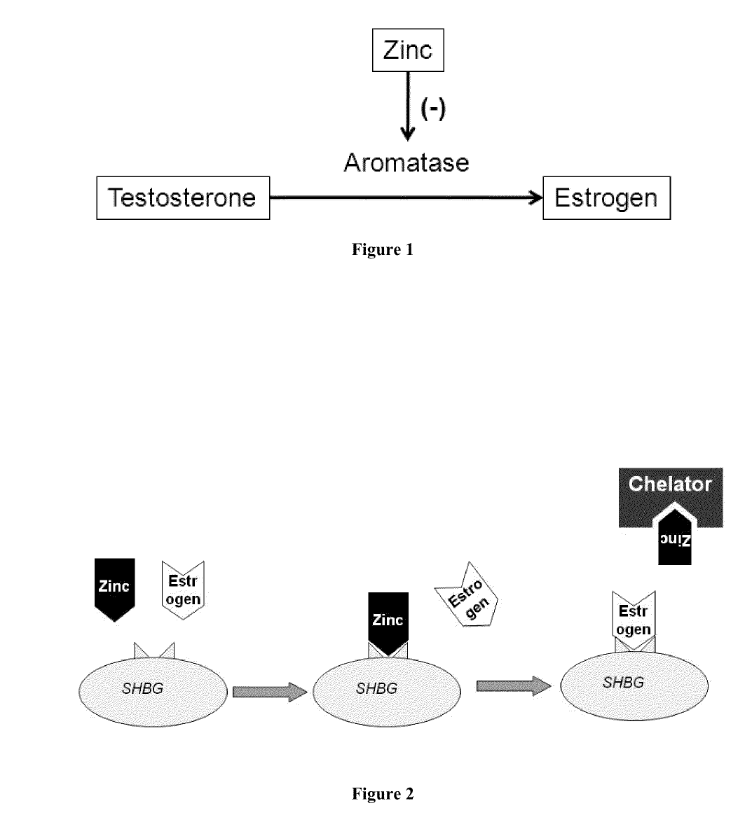

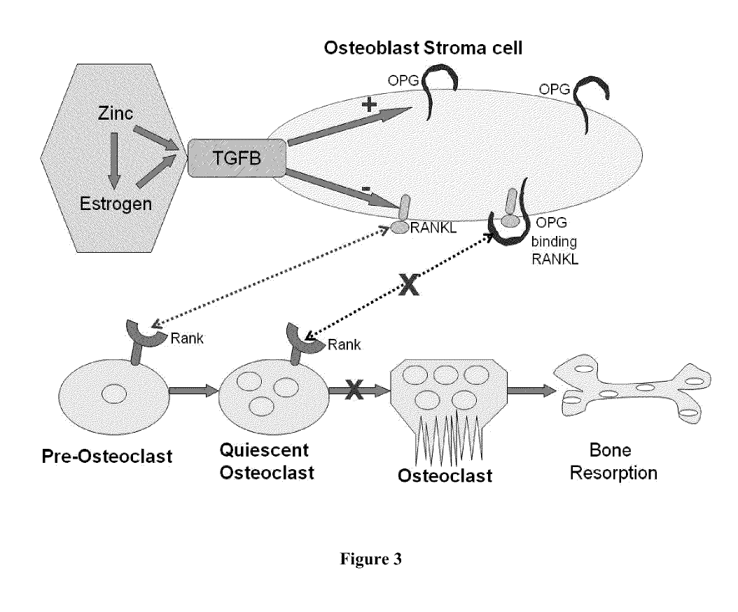

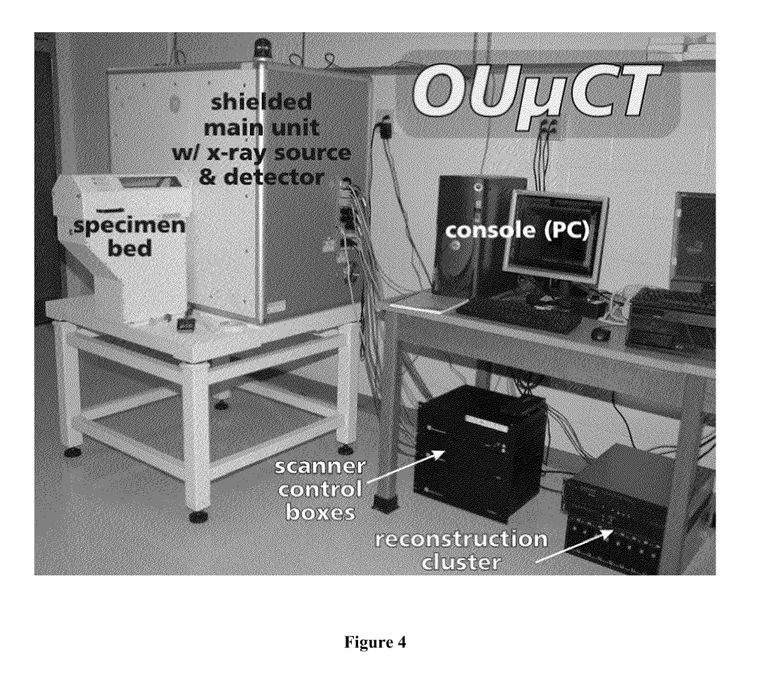

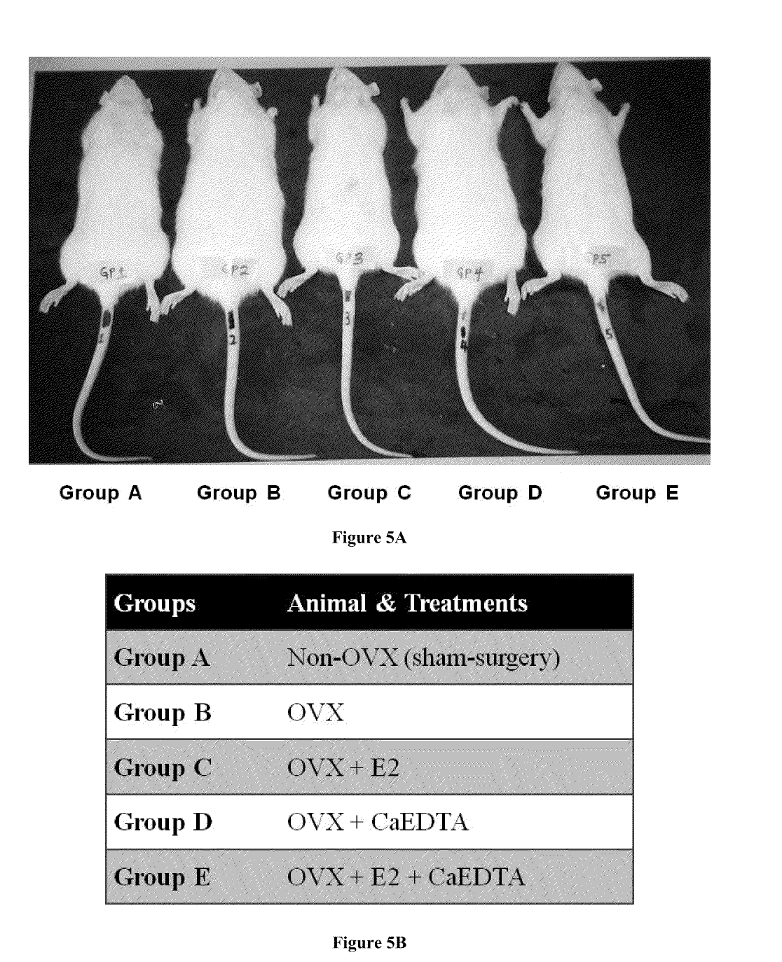

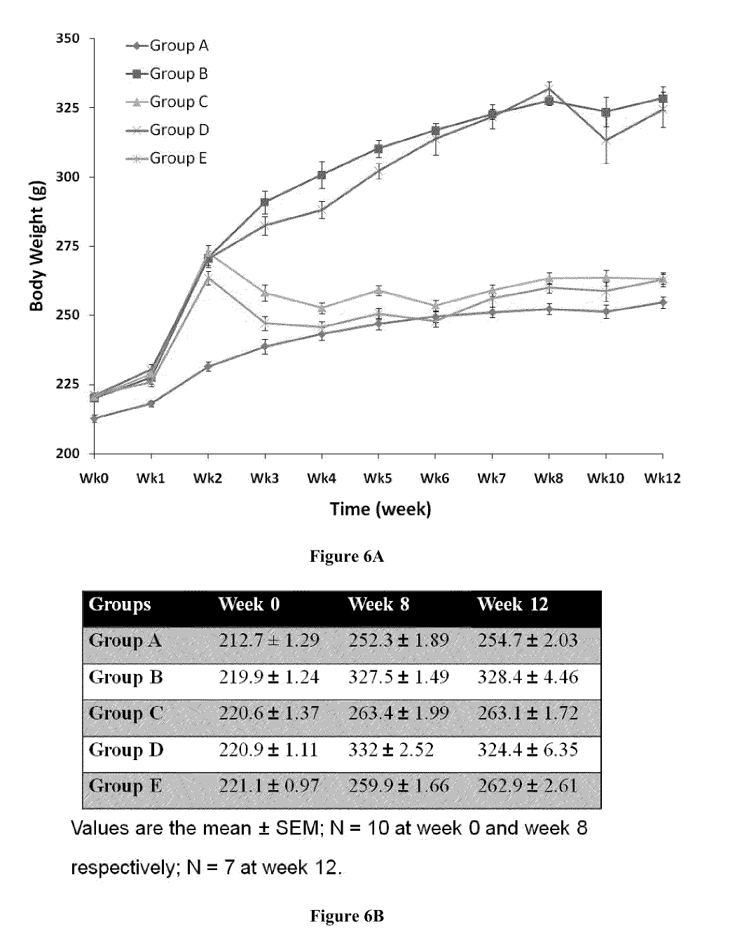

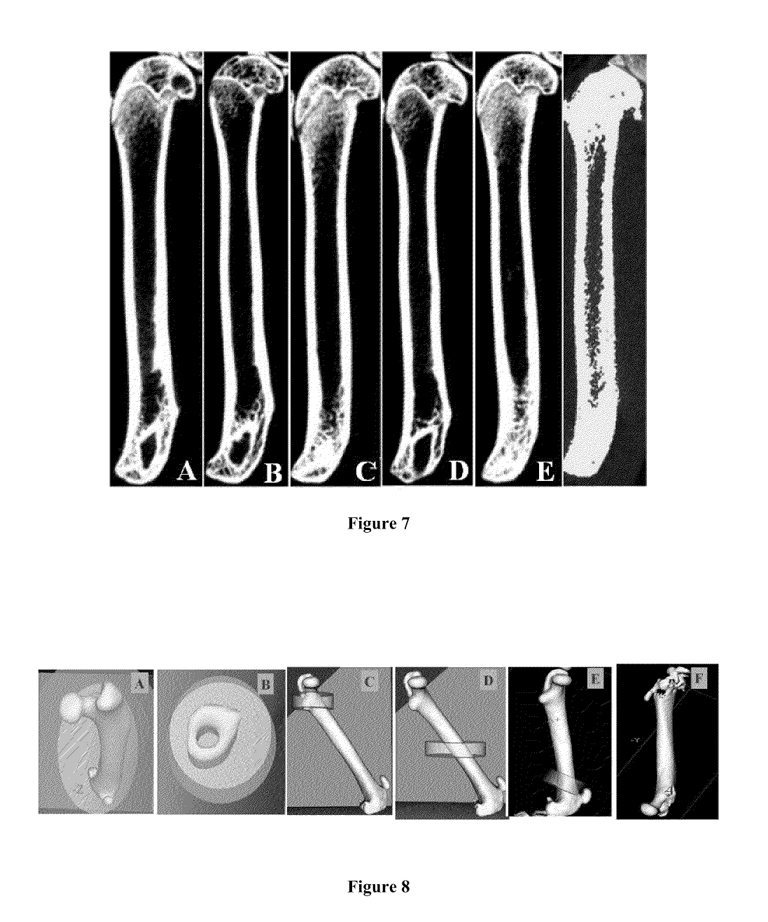

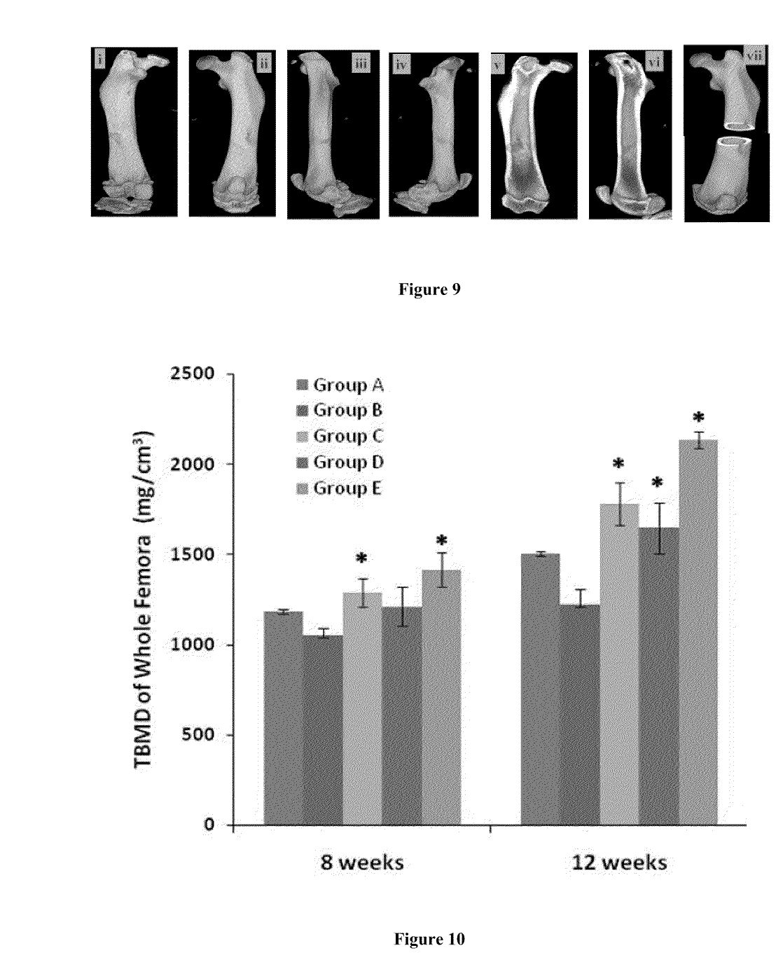

### DETAILED DESCRIPTION

The present invention will now be described by reference to some more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

In one embodiment, bone loss in an individual is treated or prevented with any physiologically suitable Zn²⁺ chelator. Suitable chelators include Clioquinol (iodochlorhydroxyquin), N,N,N′,N′-tetrakis(2-pyridylmethyl)ethylenediamine (TPEN), Dipicolinate (pyridine2,6-dicarboxylic), Diethyldithiocarbamate (DEDTC), Diethylenetriaminepentacetic acid, (DTPA), tetracycline, Calcium ethylenediaminetetra-acetic acid (CaEDTA), ethylenediaminetetra-acetic acid (EDTA), ethylene glycol tetraacetic acid (EGTA), aminophenol triacetic acid (APTRA), and 1,2-bis(o-aminophenoxy)ethane-N,N,N′,N′-tetraacetic acid (BAPTA). Clioquinolis a metal-protein-attenuating compound (MPAC) bioavailable Cu/Zn chelator. It is an anti-bacterial and anti-fungal agent. N,N,N′,N′-tetrakis(2-pyridylmethyl)ethylenediamine (TPEN) is cell membrane permeable zinc chelator, a transition metal chelator which improves myocardial protection during prolonged ischemia, and a chelator with strong affinities for Zn2+, Fe2+, and Mn2+, and low affinity for Ca2+. Dipicolinate(pyridine2,6-dicarboxylic) is zinc chelator. (Diethyldithiocarbamate (DEDTC) is a cell membrane permeable zinc chelator. Diethylenetriaminepentacetic acid, (DTPA) is a cell membrane impermeable zinc chelator. Antibiotic drugs of the tetracycline family (group) are chelators of Zn²⁺ ions. Calcium ethylenediaminetetra-acetic acid (EDTA) is a cell membrane impermeable zinc chelator. Ethylenediaminetetra-acetic acid (EDTA), ethylene glycol tetraacetic acid (EGTA), aminophenol triacetic acid (APTRA) and 1,2-bis(o-aminophenoxy)ethane-N,N,N′,N′-tetraacetic acid (BAPTA) are the chelating agents with high affinity for zinc.

In one embodiment, bone loss in an individual is treated or prevented by further administration of an estrogen or estrogen analogue. The administration of an estrogen with a chelation agent may reduce the amount of estrogen or estrogen analogue needed to prevent bone loss. Estrogen analogues are compounds that interact with estrogen receptors and exert a similar physiological effect as estrogen. Estrogen analogues include esterified or conjugated estrogens and phytoestrogens, and Selective estrogen receptor modulators (SERMs). Commercially available estrogens and estrogen analogues include MENEST esterified estrogen, PREMPHASE estrogen, PREMPRO estrogen, PREMARIN estrogen, ESTRATAB esterified estrogen, and ESTRACE estradiol acetate.

The present invention provides methods of treating or preventing bone loss comprising administering a composition comprising a zinc chelator, or a zinc chelator and estrogen or estrogen analogue, or chelating agent and estrogen or estrogen analogue. In certain embodiments, the bone loss is associated with ankylosing spondylitis, renal osteodystrophy (e.g., in patients undergoing dialysis), osteoporosis, glucocorticoid-induced osteoporosis, Paget's disease, abnormally increased bone turnover, periodontitis, bone fractures, rheumatoid arthritis, osteoarthritis, periprosthetic osteolysis, osteogenesis imperfecta, metastatic bone disease, hypercalcemia of malignancy, multiple myeloma, bone loss associated with microgravity, Langerhan's Cell Histiocytosis (LHC), bone loss associated with renal tubular disorders, or bone loss associated with bed-ridden conditions.

In one embodiment, the compositions are administered to treat patients with all forms of osteoporosis. Osteoporosis is a systemic skeletal disease characterized by low bone mass and microarchitectural deterioration of bone tissue, with a consequent increase in bone fragility. Consequently, many individuals, both male and female, experience pain, disability, and diminished quality of life (QOL) caused by osteoporosis. The World Health Organization (WHO) has established the following definitions of osteoporosis based on bone mass density measurements in white women: Normal—Bone density no lower than 1 standard deviation (SD) below the mean for young adult women (T-score above −1); Low bone mass (osteopenia)—Bone density 1.0-2.5 SD below the mean for young adult women (T-score between −1 and −2.5); Osteoporosis—Bone density 2.5 SD or more below the normal mean for young adult females (T-score at or below −2.5).

Osteoporosis has been divided into several classifications according to etiology and localization in the skeleton. Osteoporosis initially is divided into localized and generalized categories. These main categories are classified further into primary and secondary osteoporosis.

Primary osteoporosis occurs in patients in whom a secondary cause of osteoporosis cannot be identified, including juvenile and idiopathic (type I and type H) osteoporosis. Juvenile osteoporosis usually occurs in children or young adults (approx 8-14 years old) of both sexes. These patients have normal gonadal function. The first sign of juvenile osteoporosis is usually pain in the lower back, hips, and feet, often accompanied by difficulty walking. There may also be knee and ankle pain and fractures of the lower extremities. Physical malformations also may be present. These include abnormal curvature of the upper spine (kyphosis), loss of height, a sunken chest, or a limp. These physical malformations are sometimes reversible after the juvenile osteoporosis has run its course. Type I osteoporosis (postmenopausal osteoporosis) usually occurs in women aged 50-65 years. This type of osteoporosis is characterized by a phase of accelerated bone loss, primarily from trabecular bone. In this phase, fractures of the distal forearm and vertebral bodies are common. Type II osteoporosis (age-associated or senile) occurs in both women and men older than 70 years. This form of osteoporosis represents bone loss associated with aging. Fractures comprise both cortical and trabecular bone. In addition to wrist and vertebral fractures, hip fractures often are seen in type II osteoporosis.

Secondary osteoporosis occurs when an underlying disease or chronic condition causes osteoporosis. This includes endogenous and exogenous thyroxine excess, hyperparathyroidism, malignancies, gastrointestinal diseases, medications, renal failure and connective tissue diseases, bone marrow disease, immobilization, and drug use. Even the clinical history may not be completely revealing, as a patient with known metastatic disease can develop compression fractures from osteoporosis secondary to chemotherapy or administration of steroids, and radiation therapy can weaken the bone.

In another embodiment, the compositions are used to treat subjects who do not yet have osteoporosis, but who are at risk for getting osteoporosis, such as post-menopausal women, patients with osteopenia (mild thinning of the bone mass), subjects with chronic inflammatory joint diseases (described below) or people who are over the age of 70. Osteopenia results when the formation of bone (osteoid synthesis) is not enough to offset normal bone loss (bone lysis).

In another embodiment, the bone density of an individual may be determined by any suitable method known in the art. Such suitable methods include densitometry scans, for example dual energy X-ray absorptiometry scans, of the lumbar spine, hip or forearm.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a zinc chelator, or a zinc chelator and estrogen or estrogen analogue, or a chelating agent and estrogen or estrogen analogue, and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. The excipients can be chosen, for example, to effect delayed release of an agent or to selectively target one or more cells, tissues or organs. The pharmaceutical composition can be in dosage unit form such as tablet, capsule, sprinkle capsule, granule, powder, syrup, suppository, injection or the like. In another embodiment, the composition is present in a transdermal delivery system, e.g., a skin patch.

In some embodiments, a pharmaceutically acceptable carrier contains physiologically acceptable agents that act, for example, to stabilize or to increase the absorption of a compound such as a zinc chelator, estrogen or estrogen analogue. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a compound of the invention.

Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, a pharmaceutical composition (preparation) is administered to a subject by any of a number of routes of administration including, for example, orally (for example, drenches as in aqueous or non-aqueous solutions or suspensions, tablets, boluses, powders, granules, pastes for application to the tongue); sublingually; anally, rectally or vaginally (for example, as a pessary, cream or foam); parenterally (including intramusclularly, intravenously, subcutaneously or intrathecally as, for example, a sterile solution or suspension); nasally; subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water.

In some embodiments, the formulations are conveniently presented in unit dosage form or prepared by any methods well known in the art of pharmacy. The amount of active ingredient which can be combined with a carrier material to produce a single dosage form will vary depending upon the host being treated, and the particular mode of administration.

The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a zinc chelator, estrogen or estrogen analogue, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In some embodiments, formulations of the invention suitable for oral administration are in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. In some embodiments, solid compositions of a similar type are employed as fillers in soft and hard-filled gelatin capsules using such excipients as lactose or milk sugars, as well as high molecular weight polyethylene glycols and the like.

In one embodiment, a tablet is made by compression or molding, optionally with one or more accessory ingredients. In another embodiment, compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. In still another embodiment, molded tablets are made by molding in a suitable machine a mixture of the powdered compound moistened with an inert liquid diluent.

In other embodiments, the tablets, and other solid dosage forms of the pharmaceutical compositions, such as dragees, capsules, pills and granules, are optionally scored or prepared with coatings and shells, such as enteric coatings and other coatings well known in the pharmaceutical-formulating art. In some embodiments, they are formulated so as to provide slow or controlled release of the active ingredient therein using, for example, hydroxypropylmethyl cellulose in varying proportions to provide the desired release profile, other polymer matrices, liposomes and/or microspheres. In some embodiments, they are sterilized by, for example, filtration through a bacteria-retaining filter, or by incorporating sterilizing agents in the form of sterile solid compositions that can be dissolved in sterile water, or some other sterile injectable medium immediately before use. In some embodiments, these compositions contain opacifying agents and may be of a composition that releases the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

In some embodiments, the pharmaceutical composition is a suspension which, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In other embodiments, formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

In some embodiments, formulations of the pharmaceutical compositions for administration to the mouth are presented as a mouthwash, or an oral spray, or an oral ointment.

Dosage forms for the topical or transdermal administration include powders, sprays, ointments, pastes, creams, lotions, gels, solutions, patches and inhalants. In some embodiments, the active compound may be mixed under sterile conditions with a pharmaceutically acceptable carrier, and with any preservatives, buffers, or propellants that are required.

In some embodiments, the ointments, pastes, creams and gels contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound of the present invention to the body. Such dosage forms can be made by dissolving or dispersing the active compound in the proper medium. In some embodiments, absorption enhancers are used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some embodiments, these compositions also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. Some embodiments include isotonic agents, such as sugars, sodium chloride. In addition, prolonged absorption of the injectable pharmaceutical form is brought about in some embodiments by the inclusion of agents that delay absorption such as aluminum monostearate and gelatin.

In some cases, in order to prolong the effect of a drug, it is desirable to slow the absorption of the drug from subcutaneous or intramuscular injection. In some embodiments, this is accomplished by the use of a liquid suspension of crystalline or amorphous material having poor water solubility. The rate of absorption of the drug then depends upon its rate of dissolution, which, in turn, may depend upon crystal size and crystalline form.

Alternatively, delayed absorption of a parenterally administered drug form is accomplished by dissolving or suspending the drug in an oil vehicle.

In some embodiments, injectable depot forms are made by forming microencapsuled matrices of the subject compounds in biodegradable polymers such as polylactide-polyglycolide.

Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

In other embodiments, methods of introduction are provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882).

In general, a suitable daily dose of an active compound used in the compositions and methods of the invention will be that amount of the compound that is the lowest dose effective to produce a therapeutic effect. Such an effective dose will generally depend upon the factors described above.

In one embodiment, the dosage of a zinc chelator for a human is 100 mg to 3000 mg/day, more preferably 250 mg to 1000 mg/day. In one embodiment, the dosage is 50-200 mg/ml of Edtate Calcium Disodium in a 5 ml injection.

In one embodiment, the dosage of an estrogen or estrogen analogue for a human is 0.15 mg/day to 1.2 mg/day. For example, the dosage may be 0.3, 0.45, or 0.625 mg/day. In one embodiment of the invention, the administration of a zinc chelator reduces the amount of estrogen or estrogen analogue needed to prevent bone loss, in comparison to treatment with estrogen or an estrogen analogue alone.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily.

In some embodiments, the active compound will be administered once daily.

The patient receiving this treatment is any animal in need, including primates, in particular humans, and other mammals such as equines, cattle, swine and sheep; and poultry and pets in general.

In certain embodiments, a composition comprising a zinc chelator, or a zinc chelator and estrogen or estrogen analogue, or a chelating agent and estrogen or estrogen analogue is used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds.

In one embodiment, the method of treating bone loss comprises administering a composition of a zinc chelator, or a zinc chelator and estrogen or estrogen analogue, conjointly with an additional agent useful in the treatment of bone loss. In certain embodiments, the composition of a zinc chelator, or a zinc chelator and estrogen or estrogen analogue, is conjointly administered with a bisphosphonate (e.g., ibandronate, zolendronate, risedronate, etidronate, or alendronate), a selective estrogen receptor modulator (e.g., raloxifene), or hormone treatment (e.g., calcitonin or teriparitide). In certain embodiments, the composition of a zinc chelator, or a zinc chelator and estrogen or estrogen analogue, is conjointly administered with growth factors or other therapeutic agents that have a positive effect on the growth of bone or connective tissue, such as osteoprotegerin, interleukins, MMP inhibitors, beta glucans, integrin antagonists, calcitonin, proton pump inhibitors, protease inhibitors, insulin-like growth factor-1, platelet-derived growth factor, epidermal growth factor, inhibitors of transforming growth factor-alpha, transforming growth factor-beta, bone morphogenetic protein, parathyroid hormone, osteoprotegerin, a fibroblast growth factor, Vitamin D, vitronectin, plasminogen-activator inhibitor, or a protease inhibitor such as a metalloprotease inhibitor, or compounds known to be beneficial to bone formation, such as calcium, fluoride, magnesium, boron, or a combination thereof.

In embodiments where a combination of a zinc chelator and estrogen (or estrogen analogue), are administered, the zinc chelator and estrogen (or estrogen analogue) can be administered simultaneously, e.g., as a single formulation comprising both components or in separate formulations, or can be administered at separate times, provided that, at least at certain times during the therapeutic regimen, both the zinc chelator and estrogen (or estrogen analogue) are present simultaneously in the patient at levels that allow a therapeutically favorable effect.

In embodiments where a combination of a chelating agent and estrogen (or estrogen analogue), are administered, the chelating agent and estrogen (or estrogen analogue) can be administered simultaneously, e.g., as a single formulation comprising both components or in separate formulations, or can be administered at separate times, provided that, at least at certain times during the therapeutic regimen, both the chelating agent and estrogen (or estrogen analogue) are present simultaneously in the patient at levels that allow a therapeutically favorable effect.

This invention includes the use of pharmaceutically acceptable salts of the active compounds in the compositions and methods of the present invention. In certain embodiments, contemplated salts of the invention include alkyl, dialkyl, trialkyl or tetra-alkyl ammonium salts. In certain embodiments, contemplated salts of the invention include Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In another embodiment, other disease states are treated or prevented by the administration of a zinc chelator, or a zinc chelator and estrogen, or a chelating agent and estrogen. Such diseases and treatments include Alzheimer's disease (AD), treated, for example, with Clioquinol; breast cancer, treated, for example, with TPEN or DTPA; sterility, treated, for example, with DEDTC or CaEDTA; and Myocardial Ischemia, treated, for example, with TPEN.

All scientific publication and/or patents cited herein are specifically incorporated by reference. The following examples illustrate various aspects of the invention but are in no way intended to limit the scope thereof.

### EXAMPLES

An objective of this research was to understand the effect of a zinc chelator CaEDTA by itself and in combination with estrogen (17B-estradiol) (E2) on bone mineral density in estrogen deficient female rats. The ovariectomized rat (OVX) is a useful model of ovarian deficient osteopenia, replicating many aspects of osteoporosis.

Our results are consistent with previous studies that the estrogen treatment improves tissue bone mineral density (TBMD) in estrogen deprived rats (p<0.05). Ovariectomies reduced TBMD in the whole femur and in the femoral regions that are enriched in trabecular bone. Treatment with CaEDTA or estrogen negates the detrimental effects induced by ovariectomies. Major findings here are that CaEDTA administration significantly increases TBMD of femora in OVX, and normalize TBMD to the level shown in sham control. Furthermore, the co-administration of CaEDTA and estradiol give higher TBMD than the estradiol alone indicating the addition/supplement interaction of two treatments. We have concluded that CaEDTA alone or in combination with estrogen can effectively prevent the decrease in TBMD.

This study indicates that the alteration of zinc homeostasis affects bone mineral density (BMD) in estrogen deprived rats. The application of zinc chelator CaEDTA may increase availability of estrogen through removing the inhibition of zinc in estrogen conversion. Unlike estrogen, the treatment of CaEDTA had no effect on body weight. The different action in body weight suggests that CaEDTA may act at different mechanism from estrogen.

One mechanism we propose here is that zinc chelation treatment increases parathyroid hormone, which improves bone quality and reduces bone loss. Studies have shown that zinc can negatively regulate the availability of parathyroid hormone.

Another mechanism we propose here is that zinc chelation treatment increases the availability of estrogen by preventing the metabolism of estrogen. Studies have also shown zinc's ability to compete with the estrogen transport via sex hormone-binding globulin (SHBG), a protein that transports estrogen in the blood. SHBG has been shown to have specific binding sites for zinc. Zinc binding to a site at the entrance of the steroid-binding pocket in human SHBG has been shown to reduce the affinity of SHBG for estrogen. Zinc chelating agent removes zinc from SHBG and allots the binding site to estrogen. This mechanism can be significant since estrogen is under the extensive first-pass metabolism which dramatically reduces the bioavailability of administrated estrogen. SHBG bound estrogen may not only reduce the first-pass metabolism but also lessen the renal secretion. The administration of estrogen in combination with a zinc chelating agent yield more active estrogen in the system.

### Subjects Human, and Animals

Of fifty 8-week-old female Sprague-Dawley rats, forty were ovariectomized and ten non-ovariectomized. The rats received a standard diet of rodent chow (12-15 g/d) and water ad libitum. All rats were kept on an alternating 12-hour-light and 12-hour-dark cycle. The temperature inside the chambers was 22° C. (±2) with relative humidity from 40 to 60%. After the seven days acclimatization period, the 40 ovariectomized rats were randomly divided in four groups of 10 rats.

### Drug Administration

Group A (Non-OVX) was normal Control (no drug administration); Group B (OVX) is OVX control (no drug administration); Group C (OVX+E2) were administered 200 μg/kg/day 17β-estradiol (E2); Group D (OVX+CaEDTA) were administered 200 mg/kg/day CaEDTA intraperitoneal; Group E (OVX+E2+CaEDTA) were administered 200 μg/kg/day 17β-estradiol (E2) subcutaneously plus 200 mg/kg/day CaEDTA intraperitoneal at the same time. CaEDTA a broad-spectrum divalent-cation chelator that binds zinc with higher affinity than Calcium (Bers et al., 1994). All experiments were performed at the same time twice every week and in the light period. Rat body weights were determined weekly, animal care was provided in accordance with a protocol approved by the Local Animal Care Committee (Ohio University).

### Experiment

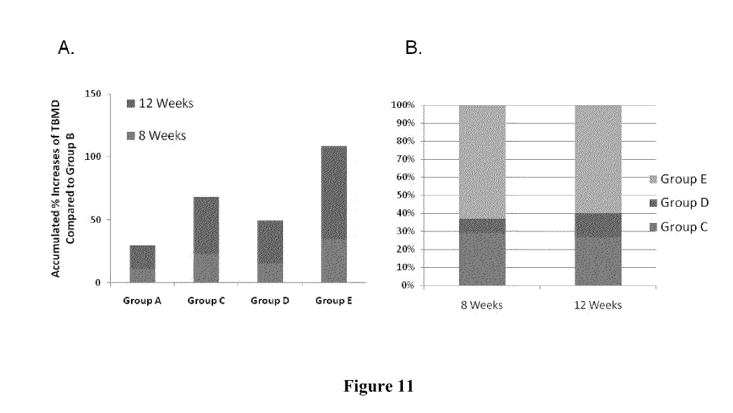

The rats were weighed and euthanized with carbon dioxide. The left femur bones were removed and stored in a −80° C. freezer. The bone mineral density was analyzed using microcomputer tomography (microCT). The bone mineral density of the five different rat groups was assayed in various region of the femur bone, including the midshaft femur, the proximal femur, the distal femur, and the entire femur. We measured Tissue Bone Mineral Density (TBMD), which can be manually set to include only bone. Therefore, TBMD is a parameter output by the BMD utility in our analysis. As indicated in FIGS. 10 and 11, the measured bone mineral densities show that the OVX rats had reduced density in the whole femur, compared to the Non-OVX control rats. The data also indicate that treatment of the OVX rats with E2, CaEDTA, or E2 and CaEDTA negates the detrimental effects induced by ovariectomies.

What is claimed is:

1. A method of treating or preventing bone loss in a subject, comprising providing a therapeutically effective amount of at least one chelating agent.

2. The method according to claim 1, wherein the at least one chelating agent is a zinc chelator.

3. The method according to claim 2, wherein the dosage of the zinc chelator is 100 mg to 3000 mg per day.

4. The method according to claim 2, wherein the zinc chelator is at least one of clioquinol (iodochlorhydroxyquin), N,N,N′,N′-tetrakis(2-pyridylmethyl)ethylenediamine (TPEN), dipicolinate (pyridine2,6-dicarboxylic), diethyldithiocarbamate (DEDTC), diethylenetriaminepentacetic acid, (DTPA), tetracycline, calcium ethylenediaminetetra-acetic acid (CaEDTA), ethylenediaminetetra-acetic acid (EDTA), ethylene glycol tetraacetic acid (EGTA), aminophenol triacetic acid (APTRA), and 1,2-bis(o-aminophenoxy)ethane-N,N,N′,N′-tetraacetic acid (BAPTA).

5. The method according to claim 2, further comprising providing to said subject a therapeutically effective amount of estrogen or at least one estrogen analogue.

6. The method according to claim 5, wherein the dosage of the estrogen or at least one estrogen analogue is 0.15 mg to 1.2 mg per day.

7. The method according to claim 5, wherein the at least one estrogen analogue is selected from the group consisting of esterified estrogens, conjugated estrogens, phytoestrogens, and selective estrogen receptor modulators (SERMs).

8. The method according to claim 5, wherein the estrogen or at least one estrogen analogue is selected from the group consisting of MENEST esterified estrogen, PREMPHASE estrogen, PREMPRO estrogen, PREMARIN estrogen, ESTRATAB esterified estrogen, and ESTRACE estradiol acetate.

9. The method according to claim 5, further comprising providing to said subject at least one of a bisphosphonate, a selective estrogen receptor modulator, or a hormone.

10. The method according to claim 1, wherein the bone loss to be treated or prevented is at least one of ankylosing spondylitis, renal osteodystrophy, osteoporosis, glucocorticoid-induced osteoporosis, Paget's disease, abnormally increased bone turnover, periodontitis, bone fractures, rheumatoid arthritis, osteoarthritis, periprosthetic osteolysis, osteogenesis imperfecta, metastatic bone disease, hypercalcemia of malignancy, multiple myeloma, bone loss associated with microgravity, Langerhan's Cell Histiocytosis (LHC), bone loss associated with renal tubular disorders, or bone loss associated with bed-ridden conditions.

11. The method according to claim 1, wherein the subject is a human.

12. The method according to claim 11, wherein the human is at risk for getting osteoporosis, diagnosed with osteopenia, diagnosed with chronic inflammatory joint diseases, or is over the age of 70.

13. A composition for the treatment or prevention of bone loss in a subject comprising:
at least one chelating agent; and
estrogen or at least one estrogen analogue.

14. The composition according to claim 13, wherein the at least one chelating agent is a zinc chelator.

15. The composition according to claim 14, wherein the zinc chelator is at least one of clioquinol (iodochlorhydroxyquin), N,N,N′,N′-tetrakis(2-pyridylmethyl)ethylenediamine (TPEN), dipicolinate (pyridine2,6-dicarboxylic), diethyldithiocarbamate (DEDTC), diethylenetriaminepentacetic acid, (DTPA), tetracycline, calcium ethylenediaminetetra-acetic acid (CaEDTA), ethylenediaminetetra-acetic acid (EDTA), ethylene glycol tetraacetic acid (EGTA), aminophenol triacetic acid (APTRA), and 1,2-bis(o-aminophenoxy)ethane-N,N,N′,N′-tetraacetic acid (BAPTA).

16. The composition according to claim 13, wherein the at least one estrogen analogue is selected from the group consisting of esterified estrogens, conjugated estrogens, phytoestrogens, and selective estrogen receptor modulators (SERMs).

17. The composition according to claim 16, wherein the estrogen or at least one estrogen analogue is selected from the group consisting of MENEST esterified estrogen, PREMPHASE estrogen, PREMPRO estrogen, PREMARIN estrogen, ESTRATAB esterified estrogen, and ESTRACE estradiol acetate.

18. The composition according to claim 13, further comprising at least one of a bisphosphonate, a selective estrogen receptor modulator, or a hormone.

19. The composition according to claim 13, further comprising a pharmaceutically acceptable carrier.

20. The composition according to claim 19, further comprising an adjuvant.
